(19) United States
(12) Patent Application Publication
(10) Pub. No.: US 2012/0109140 A1
(43) Pub. Date: May 3, 2012

(54) Implant Instrument and Guide System for the Implant Instrument

(75) Inventor: Isao Akutsu, Tokyo (JP)

(73) Assignee: Implantdent Co., Ltd., Tokyo (JP)

(21) Appl. No.: 13/261,141

(22) PCT Filed: Aug. 25, 2010
(86) PCT No.: PCT/JP2010/064345

§ 371 Date: Jan. 12, 2012

(30) Foreign Application Priority Data
Sep. 1, 2009 (JP) 2009-201263

(51) Int. Cl. A61B 17/56

(52) U.S. Cl. 606/96; 606/86 R

(57) ABSTRACT

A guide system for implant instrument has a guide ring attached to the surgical guide supported by teeth, etc. and a ring guide body attached to a drill for forming an implant hole in jawbone. The guide ring is provided with a slit portion on its side face through which a drill bur is inserted from its lateral side at the time of drilling a hole, and the ring guide body of the drill is guided into the guide ring for formation of an implant hole.

### TECHNICAL FIELD

The present invention relates to an implant instrument and a guide system for implant instrument, suitable to stably, securely and accurately form a hole for a dental implant at a predetermined position and to accurately insert an implant into the predetermined position.

### BACKGROUND ART

In recent years, a dental treatment to set a denture after placing an implant (artificial tooth root) in a site of tooth missed is performed. In this treatment, an implant hole is formed at a site of a lost tooth by use of a drill attached to a drilling device such as a handpiece, and at this time, a surgical guide is usually employed to drill the implant hole in order to guide the drill so that the implant hole would be formed at a predetermined position and in a predetermined direction.

Into this surgical guide, a metallic guide ring is fitted to guide the drill through the surgical guide supported by the jawbone, etc.

As a method for drilling holes by use of this guide ring, there has been known a method wherein the drilling is performed while a drill bur is brought into directly contact with the inner face of the guide ring, and a method as described in Japanese Patent No. 3793603 wherein a guide member is attached to a drilling device such as a handpiece, this guide member is positioned around a drill which is to be attached to the drilling device so that the guide member would not be in contact with the drill, and the guide member is guided by the guide ring to perform the drilling.

### RELATED ART DOCUMENTS

### Patent Documents

Patent Document 1: Japanese Patent No. 3793603

### SUMMARY OF THE INVENTION

### Problems to be Solved by the Invention

The type wherein a drill bur is brought into directly contact with the guide ring, skill is demanded for drilling, and if the drilling device is only slightly inclined, the drill bur may be in contact with the inner face of the guide ring and shave off its inside, and such shavings are likely to enter into the implant hole of the jawbone, which results in adverse affects at a connecting area with the implant or the drilling direction may sometimes be tilted. Further, in case the drill bur is to pass directly through the guide ring, only drills with same diameter can be used for one type of surgical guide, accordingly several surgical guides should be prepared corresponding to the diameters of drills, such being uneconomical.

Furthermore, at the time of drilling, a frictional heat is generated between the guide ring and the drill bur, and this frictional heat damages the guide ring and the drill bur, and at the same time, may adversely affect the jawbone.

Further, the method by which the guide member is directly attached to the handpiece, the device being bulky in its entirety, causes heavy workload in drilling operation and makes the operation difficult. Moreover, since the drill attached to the handpiece is inserted from the upper part of the guide ring at the time of drilling, especially in a case where the implant is inserted at the back tooth position, the drilling operation is difficult or impossible, and it is also required to make the patient open the mouth wide and suffer the patient huge distress.

Furthermore, after the implant hole is drilled, when an implant is inserted into the hole by use of an instrument such as an adaptor for implant insertion, such an instrument may deviate back and forth, and left and right, resulting in inclined placement of implants in some cases. Moreover, when an abutment, a healing cap, a healing abutment, etc. is attached to the implant by use of an instrument such as an abutment holder, a hex driver, etc., such an instrument may incline and attachment may take time in some cases.

The present invention solves such problems and provides an implant instrument and a guide system for implant instrument, by which when the implant hole is drilled into the jawbone, when an implant is inserted thereinto and when a part to be attached to the implant is installed, such operations can be safely, securely and efficiently conducted, and dental treatment can be conducted without causing heavy workload on the patient.

### Means of Solving the Problems

According to an aspect of the present invention, the guide system for implant instrument comprises a surgical guide having a guide ring, and an implant instrument such as an implant drill and an adaptor for insertion, wherein a ring guide body is provided on a shaft of the implant instrument. Further, the side face of the guide ring is provided with a slit portion, the width of which allows insertion of an implant drill bur and an implant from its lateral side and is smaller than the inner diameter of the guide ring.

According to another aspect of the present invention, the implant instrument is provided with, on its shaft, a ring guide body which is guided in the guide ring of the surgical guide.

The surgical guide having the guide ring is classified into a type wherein the surgical guide is supported by jawbone, a type wherein the surgical guide is supported by a gum, and a type wherein the surgical guide is supported by teeth. These types are generally made of a plastic material like conventional surgical guide (surgical stent). In such cases, if the surgical guide is made of a transparent material, the operation site of drilling can be observed more clearly.

When a guide ring is provided on a surgical guide, a part of the jawbone at which the implant is to be attached is subjected to CT scanning, the three dimensional image obtained by the scanning is used to determine the position, direction, etc. of the implant.

The guide ring may be made of a metal such as titanium or aluminum. When friction is generated between the guide ring and the ring guide body to be inserted thereinto, the guide ring may be made of a rigid plastic material so long as it can stand the friction. As the material, it is preferred to use a material not adversely influencing the human body.

When the outer peripheral face of the ring guide body has a circular cross section, the inner peripheral face of the guide ring is formed to have a circular cross section so that the ring guide body is movable within the guide ring in its axial direction and may be rotatably fitted therein and guided.

However, when the ring guide body is formed to be rotatable within the guide ring, the combination of cross sections is not limited thereto.

The guide ring is usually formed into a ring configuration with an inner diameter D1 of about 4 mm to 9 mm and an outer diameter D2 of about 5 mm to 10 mm, but the size is not limited thereto.

When the outer peripheral face of the ring guide body has a non-circular cross section (a polygonal shape such as ellipse, oval, quadrilateral or hexagonal, or a complex shape thereof), the inner peripheral face of the guide ring is formed to have a non-circular cross section corresponding to the outer periphery of the ring guide body so that ring guide body is movable within the guide ring in its axial direction and may be non-rotatably fitted therein and guided.

However, when the ring guide body is formed to be non-rotatable within the guide ring, the combination of cross sections is not limited thereto.

As a drill for forming the implant hole, ones with various shapes and various diameters, for example, a round bur (FIG. 11), a trephine bur (FIG. 12), a spiral drill with various diameters (FIG. 5), etc. may be used.

Other than the drills, the ring guide body may be attached to an implant instrument such as an implant adaptor, an abutment holder, a hex driver, and instruments to increase the bone width around the implant hole, such as an osteotome, a bone spreader, and a bone expander.

When the implant is inserted, as an implant insertion adaptor to be attached to the implant, a type which is used with a handpiece (FIG. 13) or a type which is handled by fingers (FIG. 14) may be used.

In addition, when the absorption of a jawbone is remarkable, the ring guide body may be attached to instruments, such as, an extension holder and a drill extension used for drilling a hole in jawbone or inserting the implant at a deep position.

At the side face of the guide ring, a slit portion may be provided so that the drill bur, the implant or the front end portion of the implant instrument may be inserted from the lateral side. In this instance, a supporting portion of the surgical guide corresponding to the slit portion is provided with a cutaway portion having a width corresponding to the slit portion or a cutaway portion expanded outwardly wider than the width of the slit portion (usually the open angle is about 30 to 60 degrees, but may be other degrees). In a case of a ring-shaped guide ring having no slit portion or in a case where the slit portion of the guide ring has no supporting portion, the cutaway portion is of course not necessary.

In general, drill burs are formed to have a diameter of about from 2 mm to 5 mm in considering the diameter of implant, and taking it into consideration, the width W of the slit portion (FIG. 2, FIG. 9 and FIG. 10) is formed to have a diameter slightly larger than the maximum diameter of the implant, i.e. usually at a level of from 4 mm to 6 mm. The width W of the slit portion is not limited to the size.

Further, the face of the slit portion may be processed so that the slit faces would be formed in parallel (FIG. 1, FIG. 2), formed straight on the same line (FIG. 10), or formed obliquely to have an appropriate open angle (in the one shown in FIG. 9, the angle is 120 degrees).

On the other hand, the ring guide body to be inserted into the guide ring has a hole to guide the shaft of the implant instrument such as a drill, and another hole to guide a drill bur or a thick shaft portion of other implant instrument. The ring guide body is supported by the thick shaft portion of the implant instrument (in some cases, the upper end portion of the drill bur corresponds to it). The ring guide body may be detachably fixed at a predetermined position of the shaft by use of a fastener.

By fixing the ring guide body with the fastener, the length of insertion of the drill bur to be inserted can be adjusted by moving the position of the ring guide body, by which it becomes possible to drill a hole in a predetermined depth depending on the length of the implant.

As the fastener, a hexagon socket head cap screw, a setscrew, etc. may be used. In any case, it is preferred that the head of the fastener does not project from the outer periphery face of the ring guide body so that the ring guide body can be smoothly guided into the guide ring.

The ring guide body may be made movable in the axial direction of the shaft of drill, etc., or rotatable relative to the shaft of drill, etc. (the cross section of the ring guide body and the shaft of e.g. a drill, etc. are formed to have a circular configuration) or non-rotatable relative to the shaft of drill, etc. (the cross section of the ring guide body and the shaft of drill, etc. are formed to have a non-circular configuration). Or, these members may be connected by screw-engagement. When both members are connected by screw-engagement, a double nut, etc. may be used to fix the ring guide body at a predetermined position.

The hole to guide the drill bur in the ring guide body may be omitted. Or this ring guide body may be integrally provided on the shaft of drill.

The diameter of the ring guide body is slightly smaller than the inner diameter D1 of the guide ring so that it can be movably guided within the guide ring, and usually formed to have a gap between both parts at a level of 0.05 mm to 0.1 mm. The ring guide body may be formed to have a size other than the size so long as the ring guide body can be smoothly slide within the guide ring and the drill and other implant instrument do not deviate within such gap. Further, in such instance, the outer diameter of the ring guide body is larger than the width W of the slit portion of the guide ring so that the ring guide body would not partly protrude or come out from the slit at the time of drilling a hole.

The ring guide body may be made of a solid material such as various types of metals and rigid plastic materials, and it is preferred to use a material having frictional resistance and not having adverse effect to human body.

At the time of drilling a hole, since the frictional heat is generated at the contact surface between the ring guide body and the guide ring (in a case where the ring guide body is fixed to the shaft of the drill), the contact surface between the ring guide body and the shaft of the drill (in a case where the ring guide body is not fixed to the shaft of the drill), and the contact surface between the drill bur and the jawbone, a water supply means is provided to cool these contact surfaces. The water supply means comprises a plurality of water supply channels in the center axis direction on the outer surface of the ring guide body so that water supply can be conducted on the contact surface between the ring guide body and the guide ring, and a plurality of water supply holes in the center axis direction in the ring guide body to cool the contact surface between the ring guide body and the drill shaft or to supply water to the outer surface of the drill bur. The number of the water supply channels and water supply holes is three or four or any other appropriate number.

After drilling a hole, when an implant is inserted into the implant hole, an implant instrument such as an adaptor for implant insertion, etc. is used. On the outer peripheral portion of the shaft of the instrument, the above ring guide body is provided. In this instance, the engagement of the implant and the implant instrument is conducted by means of a projection-and-recess engagement by a hex, a socket, etc., a screw engagement, etc. This ring guide body may be provided with the water supply channels and water supply holes.

Further, when parts such as an abutment, a healing cap or a healing abutment, is attached to the implant, an implant instrument such as an abutment holder or a hex driver is used. In this case also, on the outer peripheral surface of the shaft of instrument, the above ring guide body is provided.

When the shaft of the implant instrument such as a drill is formed to be rotatable to the ring guide body, a friction-reducing part such as an O-ring or a slide bearing is provided on the ring guide body so that the shaft can smoothly rotate.

In or On the surface of the ring guide body, a magnet may be embedded or stuck, a magnet sheet may be stuck, or a magnet layer may be provided, and the magnetic force thereof temporarily keeps the ring guide body on the handpiece when the drill is replaced.

### Effects of Invention

According to the present invention, since the ring guide body for the implant instrument such as a drill is guided into the guide ring, no deviation of the drill is caused when drilling a hole, and no misplacing is caused at the time of inserting the implant or attaching parts of implant, accordingly the drilling of holes and the placement of implant can securely be made. Further, the operation can readily be made and can be conducted rapidly and efficiently. Moreover, since a slit is formed on the guide ring, the drill bur and the implant can be inserted from the lateral side through this slit portion, and the workload and distress on patients can be reduced.

Further, when the ring guide body is fixed to the shaft of the implant instrument such as a drill by use of a fastener, the installation position of the ring guide body can easily be adjusted, and can readily be fitted to the insertion length of the drill bur or the insertion depth of the implant. Moreover, a thick shaft portion is provided on the implant instrument and the ring guide body is supported by this thick shaft portion, whereby the ring guide body can be applied to drills with various diameters of burs.

Furthermore, since the water supply holes and channels are provided in the ring guide body, the cooling water supplied by the holes and channels can cool the frictional heat generated on the contact surface between the ring guide body and the guide ring, or between the drill bur or implant and the jawbone; the cooling does not adversely affect the jawbone; and the dust formed by the frictional engagement can be washed away by the cooling water.

Further, if a magnet is placed in or on the surface of the ring guide body, or a magnet sheet is stuck, or a magnet layer is provided thereon, the ring guide body can be temporarily kept on the handpiece when the drill is replaced.

### BRIEF DESCRIPTION OF THE DRAWINGS

FIG. 1 is a perspective view of a guide ring showing an embodiment of the present invention.

FIG. 2 is a plane view of the guide ring of FIG. 1.

FIG. 3 is a plane view showing a surgical guide to which a guide ring is attached.

FIG. 4 is a plane view of a ring guide body.

FIG. 5 is a perspective view of a drill to which a ring guide body is attached.

FIG. 6 is a front view of a drill of which a ring guide body is partially cutaway.

FIG. 7 are cross-sectional views partially cutaway, showing an implant hole is drilled in a jawbone by use of a surgical guide having a guide ring and a drill having a ring guide body. (A) shows the state in which the drill is moved from the outer side toward the inner side of the lower jaw, (B) shows the state in which the drill is moved from the upper side toward the lower side, and (C) shows the state in which the drill goes down and stops.

FIG. 8 is a plane view showing a state where a surgical guide to which a guide ring is attached is mounted on the lower jaw model.

FIG. 9 is a plane view showing another embodiment of guide ring.

FIG. 10 is also a plane view showing still another embodiment of guide ring.

FIG. 11 is a front view showing another type of drill “round bur”.

FIG. 12 is a front view showing still another type of drill “trephine bur”.

FIG. 13 are front views showing an adaptor for implant insertion used when an implant is inserted into an implant hole. (A) shows a state where the adaptor for insertion is not attached to the implant, and (B) shows a state where the adaptor for insertion is attached to the implant, with a partial cross-sectional view.

FIG. 14 are front views showing a hand mount (an adaptor for implant insertion) used when an implant is inserted into the implant hole. (A) shows a state in which the mount is not attached to the implant, and (B) shows a state in which the mount is attached to the implant, with a partial cross-sectional view.

FIG. 15 is a plane view showing a guide ring of which the cross-section of the inner peripheral surface is formed in an ellipse shape

FIG. 16 is a plane view showing a ring guide body of which the cross-section of the outer peripheral surface is formed in an ellipse shape

FIG. 17 is a perspective view of a drill to which the ring guide body shown in FIG. 16 is attached.

FIG. 18 is a front view showing the drill shown in FIG. 17 with the ring guide body partially cutaway.

FIG. 19 is an enlarged cross-sectional view showing a shaft of a drill which has a projection on its periphery.

FIG. 20 are cross-sectional views showing, partially cutaway, an implant hole drilled into a jawbone by use of a surgical guide having a guide ring shown in FIG. 15 and a drill having a ring guide body shown in FIG. 16. (A) shows the state in which the drill is moved from the outer side toward the inner side of the lower jaw, (B) shows the state in which the drill is moved downward, and (C) shows the state in which a drill goes down and stops.

FIG. 21 is a plane view showing another embodiment of a ring guide body.

### MODE FOR CARRYING OUT THE INVENTION

Hereinafter, a case where an implant is placed in the back tooth area of the lower jaw will be explained.

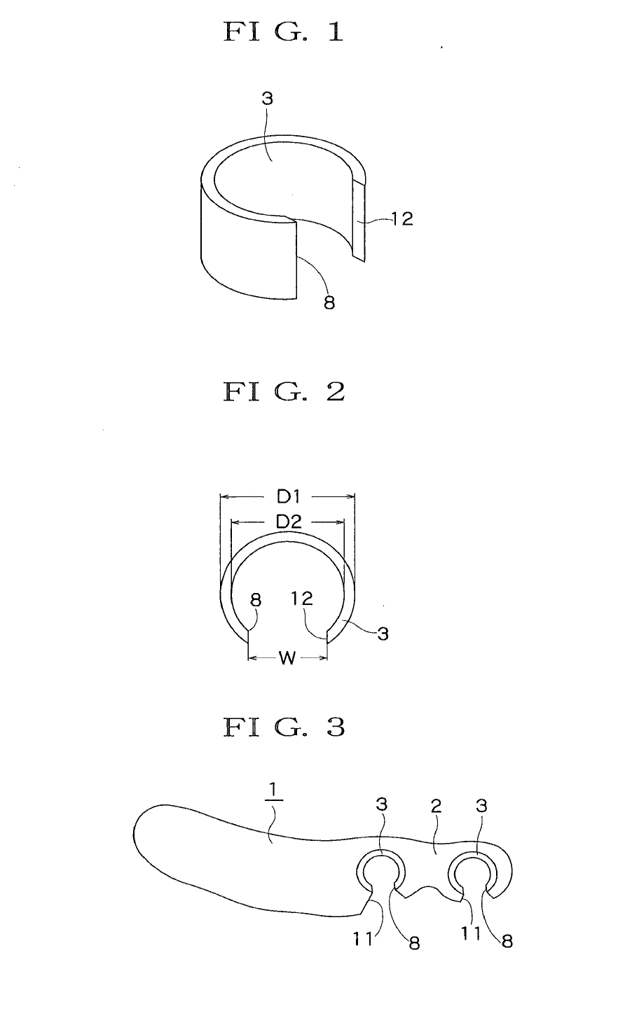

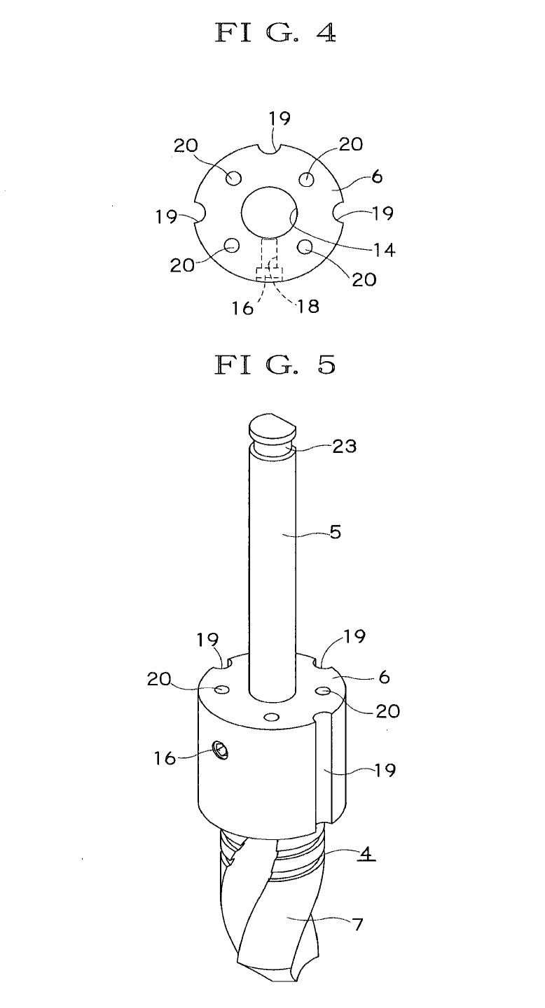

The guide system of the present invention for an implant drill, etc. has a guide ring 3 attached to a supporting portion 2 of a surgical guide 1 as shown in FIGS. 1 and 2, and further has a ring guide body 6 attached to a shaft 5 of a drill 4 as shown in FIGS. 4 to 6. On the side face of the guide ring 3, a slit 8 through which a bur 7 of the drill 4 can be inserted.

The guide ring 3 is provided at the position where the implant is placed to the supporting portion 2 of the surgical guide 1, as shown in the state of FIG. 8 wherein the surgical guide 1 abuts a jaw model 9. In this instance, the surgical guide 1 is supported by adjacent teeth 10 and the supporting portion 2 covers the surroundings of the guide ring 3, and therefore the covering portion is provided with a cutaway 11 corresponding to the width of the slit 8 of the guide ring 3.

Slit faces 12 of the guide ring 3 are formed to be in parallel to each other as shown in FIGS. 1 and 2, to have a predetermined open angle as shown in FIG. 9, or to be in parallel on the same line as shown in FIG. 10.

The ring guide body 6 attached to the drill 4 is made to fit in the guide ring 3 and to move slidingly. A tapered portion 13 is provided at the lower end portion of the ring guide body 6 so that the ring guide body 6 can be guided smoothly into the guide ring 3 when the ring guide body 6 is fitted therein. The tapered portion 13 may be provided at the upper end portion of the ring guide body 6 as well.

Inside the ring guide body 6, a hole 14 through which the drill shaft 5 is inserted and a hole 15 through which the drill bur 7 is inserted are provided. The ring guide body 6 is fixed to the shaft 5 by a fastener 16. In this instance, the length of the drill bur 7 is adjusted to be equivalent to the depth of an implant hole 17, and the ring guide body 6 is fixed at a predetermined position with the fastener 16.

In this embodiment, a hexagon socket heads cap screw is used as the fastener 16, and the hexagon socket heads cap screw is engaged with a threaded hole 18 formed in the ring guide body 6. At the time of engagement, it is preferred to insert the bolt into slightly deeper position so that the head of the bolt would not protrude from the outer periphery surface of the ring guide body 6.

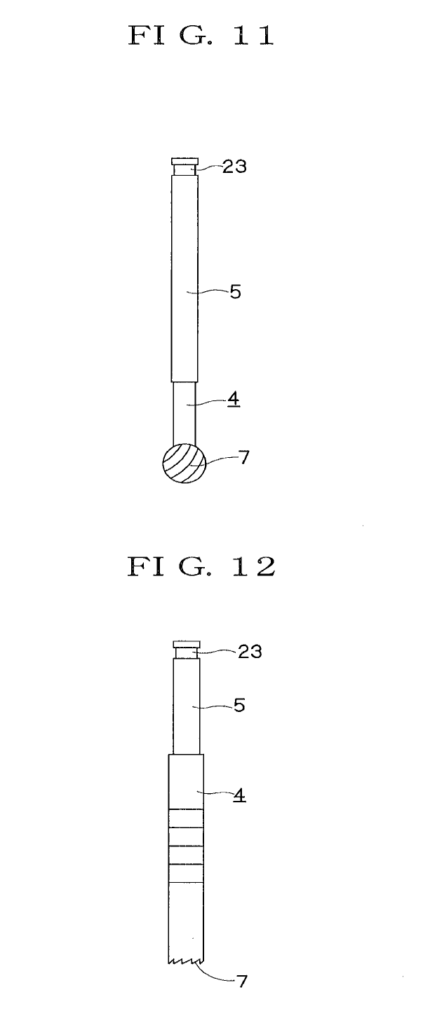

As for the type of drill to be used, various types may be used, for example, a spiral drill type as shown in FIG. 5, a round bur type as shown in FIG. 11, and a trephine bur type as shown in FIG. 12. When the ones having a straight shaft are used like the round bur shown in FIG. 11, the hole 15 of the ring guide body 6 may be omitted.

In a case where an implant hole is drilled by using this instrument, when the ring guide body 6 rotates and slides in the axial direction within the guide ring 3, frictional heat is generated at the contact surface between the ring guide body 6 and the guide ring 3. Accordingly, as shown in FIGS. 4 to 6, water supply channels 19 are provided on the outer surface of the ring guide body 6 in its axial direction, and the friction surface is cooled by supplying water through the water supply channels.

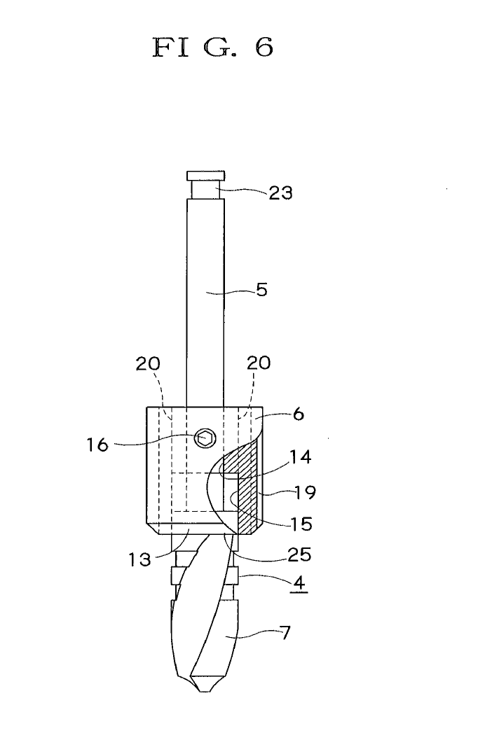

Further, at the time of drilling a hole, since frictional heat is also generated on the contact surface between the drill bur 7 and the implant hole 17, the water supply holes 20 are provided near the center of the ring guide body 6 in its axial direction (in FIG. 6, in order to readily understand the position of the water supply holes 20, the position is shifted by 45 degrees in its circumferential direction and indicated by a broken line). The friction portion between the drill bur 7 and the implant hole 17 is cooled by the water flowing through the water supply holes and along the drill bur 7.

In this embodiment, the water supply channels 19 and the water supply holes 20 are provided in the axial direction of the ring guide body 6 (in the axial direction of the drill), but instead of this direction, these may be provided, for example, in an oblique direction or in a spiral direction.

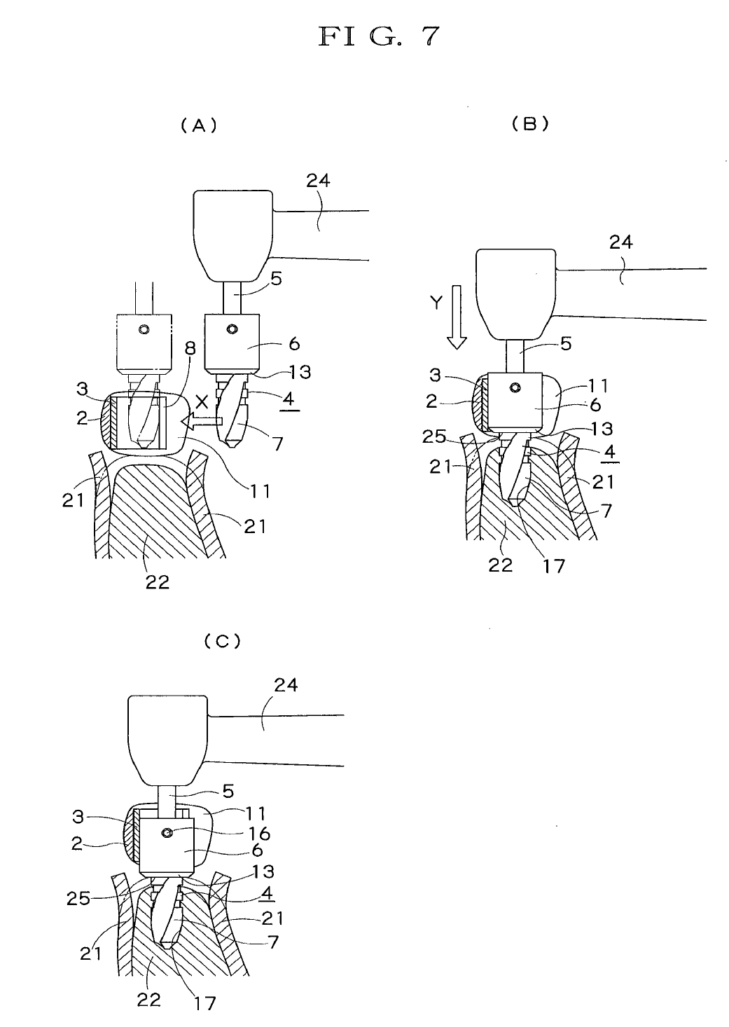

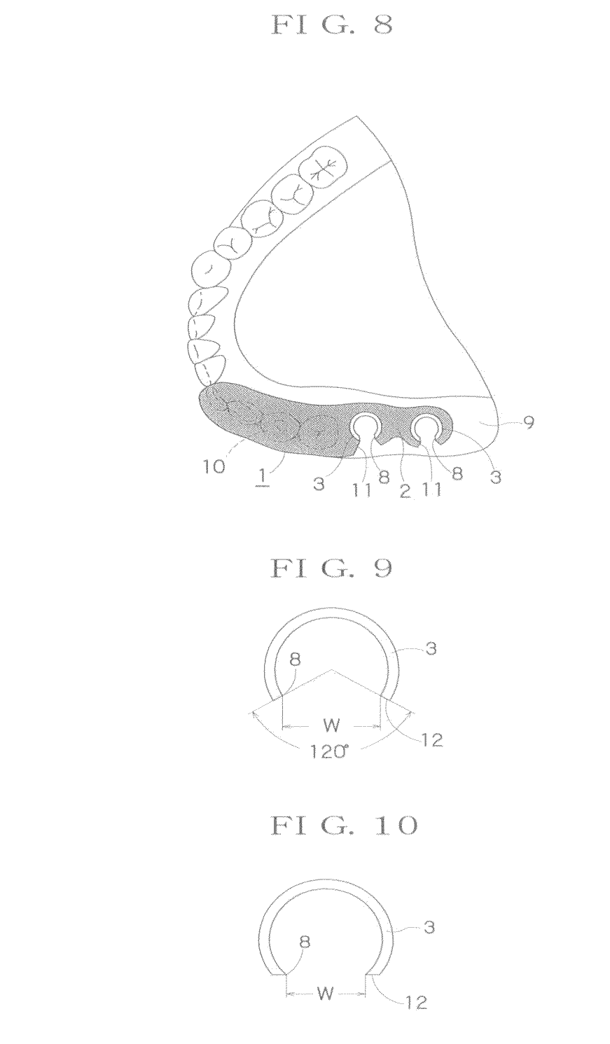

Then, a case where the implant hole 17 is drilled in the lower jaw will be explained with FIG. 7.

At first, as shown in FIG. 7(A), the gum 21 around the implant hole is opened, and a lower jawbone 22 is exposed. The surgical guide 1 is attached in such a state as shown in FIG. 8. In this instance, the surgical guide 1 is attached to the lower jaw through the adjacent teeth 10, and at the position corresponding to the implant hole, the lower face of the supporting portion 2 is not in contact with the lower jawbone 22.

In this state, using a handpiece (a hole drilling device) 24 in which the upper portion (an installation portion 23) of the shaft 5 of the drill 4 is chucked, the drill is moved in the direction of arrow X, and the drill bur 7 is guided into the guide ring 3 through the cutaway 11 of the supporting portion 2 of the surgical guide 1 and the slit 8 of the guide ring 3.

Then, as shown in FIG. 7(B), the drill 4 is moved downward in the direction of arrow Y, the ring guide body 6 which rotates with the drill 3 is guided into the guide ring 3, and the implant hole 17 is formed in the lower jawbone 22 using the drill bur 7.

Subsequently, as shown in FIG. 7(C), the drill 4 is moved downward, a lower end portion 25 of the ring guide body 6 is made to contact with the lower jawbone 22. Since the implant hole 17 is formed in the predetermined depth at this contact position, the hole drilling is stopped at this position. The stopped position can be confirmed through the slit 8 of the guide ring and the cutaway 11 of the surgical guide.

When the inner face of the guide ring 3 and the outer surface of the ring guide body 6 are formed in a tapered shape in which the upper part is wide and the lower part is narrow, the downward movement of the drill is stopped when the ring guide 6 comes into contact with the guide ring 3, and therefore this position is regarded as the stopped position of the drill.

In this embodiment, the implant hole is drilled by use of a spiral drill. However, in the beginning of the drilling, the drill as shown in FIG. 11 may be used or, if the jawbone is soft, the drill as shown in FIG. 12 or one of other drills may be used. As the drilling proceeds, the drill is replaced with one of other spiral drills having wider bur diameter to proceed the drilling.

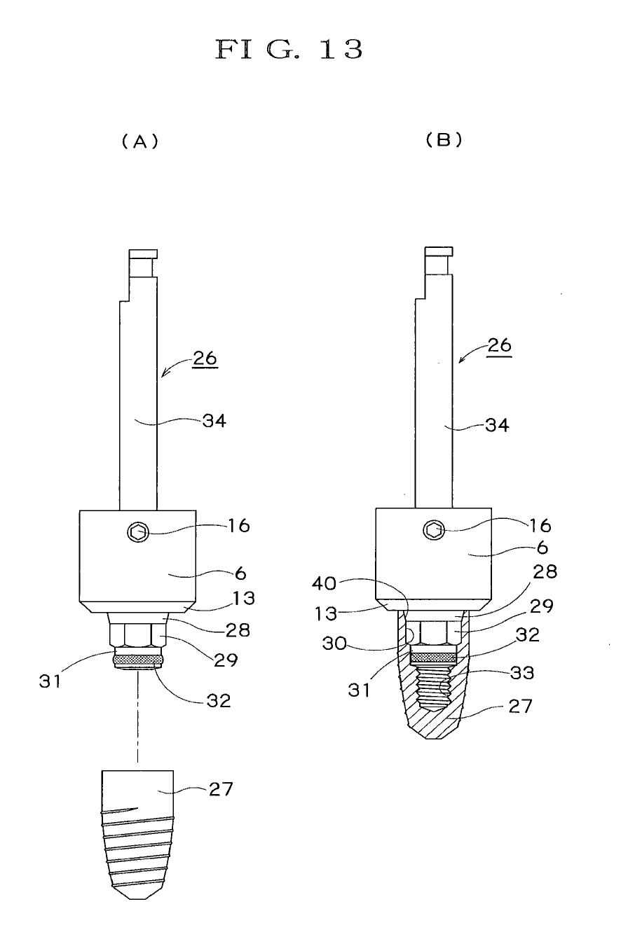

FIG. 13 shows an adaptor for implant insertion 26 used when an implant 27 is inserted into the implant hole 17 of the jawbone. In this embodiment, a projection 29 having a hexagonal cross section provided at the lower end portion of the above adaptor for implant insertion 26 via a tapered portion 28 is engaged with a tapered recess 40 and a recess 30 having a hexagonal cross section provided at the upper end portion of the implant 27. In this instance, a rubber ring 32 is attached to the outer peripheral groove of an axial end portion 31 provided below the lower end of the projection 29, and the axial end portion 31 is inserted into a threaded hole 33 of the implant to support the implant 27 by the adaptor for insertion 26.

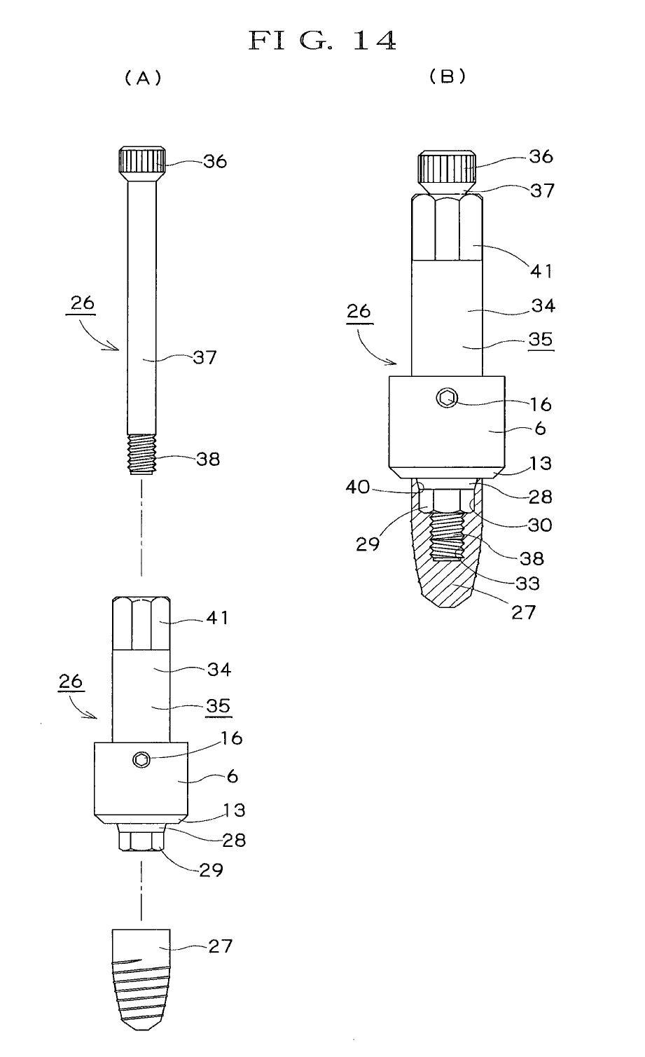

FIG. 14 shows the hand adaptor for implant insertion 26 used for insertion of the implant 27 into the implant hole 17 in the jawbone. The hand adaptor for implant insertion 26 has a mount 35 and a supporting shaft 37 having a roulette finished knob 36. The supporting shaft 37 is inserted into the mount 35 and a threaded part 38 provided at the lower end portion of the supporting shaft 37 is engaged with the threaded hole 33 formed in the implant 27 to attach the implant 27 to the adaptor for implant insertion 26. In this instance, the projection 29 having a hexagonal cross section provided at the lower end portion of the mount 35 via the tapered portion 28 is engaged with the tapered recess 40 and the recess 30 having a hexagonal cross section formed at the upper end portion of the implant 27. After the implant 27 is inserted into the implant hole 17, a wrench such as a ratchet wrench is fitted in a rotation-controlling portion 41 formed in quadrangular or hexagonal cross section at the upper part of the mount 35, and the implant 27 is rotated and threadedly inserted into the implant hole 17 in the jawbone. This adaptor for implant insertion is preferred when the jawbone is thin or the bone quality is poor, since operation can be conducted with sensitive adjustment. The threaded hole 33 formed in the implant 27 may be utilized when an abutment is attached thereto.

At the outer periphery portion of the shaft 34 of the adaptor for implant insertion 26 shown in FIGS. 13 and 14, the ring guide body 6 is provided like the case of drill. When the implant 27 is inserted into the implant hole 17 using the adaptor for implant insertion 26, the implant 27 is attached to the lower end portion of the adaptor for implant insertion 26, this implant is inserted through the slit 8 of the guide ring 3, and the adaptor for implant insertion 26 is rotated and threadedly inserted by use of a handpiece, etc. The ring guide body 6 may be provided with the fastener 16, the water supply channels 19 and the water supply holes 20 like the above case of drill. Further, when a thick shaft portion (stepped portion) is provided on the shaft 34 of the adaptor for implant insertion 26, the hole 15 is formed in the ring guide body 6 to guide the thick shaft portion like the case of the drill.

When an implant part such as an abutment, a healing cap or a healing abutment is attached to the implant 27, an implant instrument such as an abutment holder and a hex driver is used, and in such a case as well, the ring guide body is provided on the shaft of the implant instrument to attach the above part.

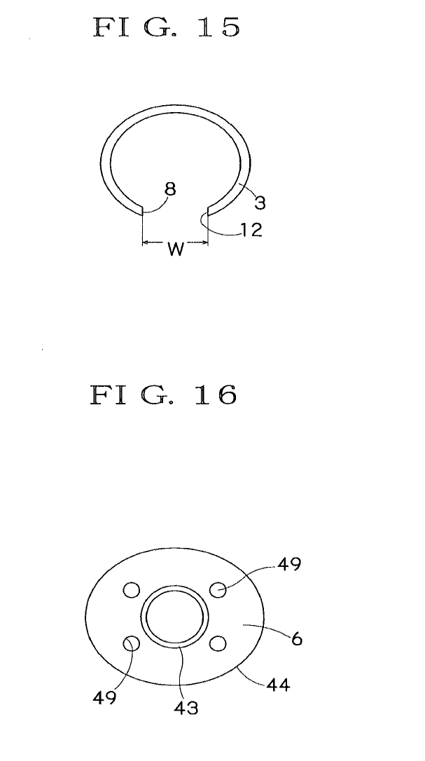

The guide ring 3 of FIG. 15 and the ring guide body 6 of FIG. 16 show other structures. The cross section of the inner peripheral surface of the guide ring 3 is formed in an ellipse configuration, and corresponding to it, the cross section of the outer peripheral surface of the ring guide body 6 is formed in an ellipse configuration.

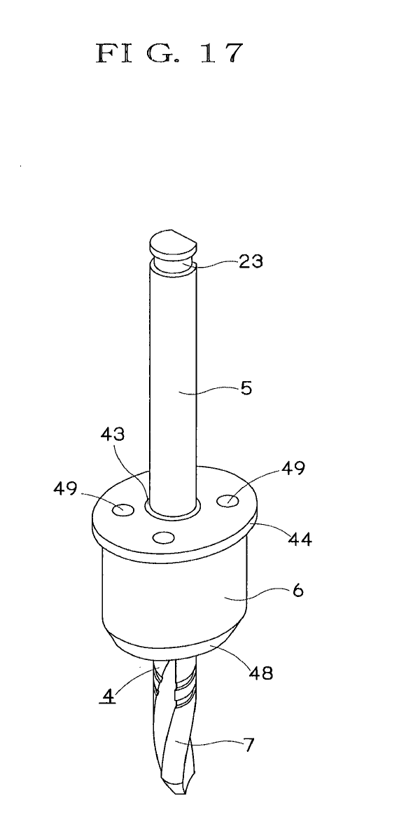

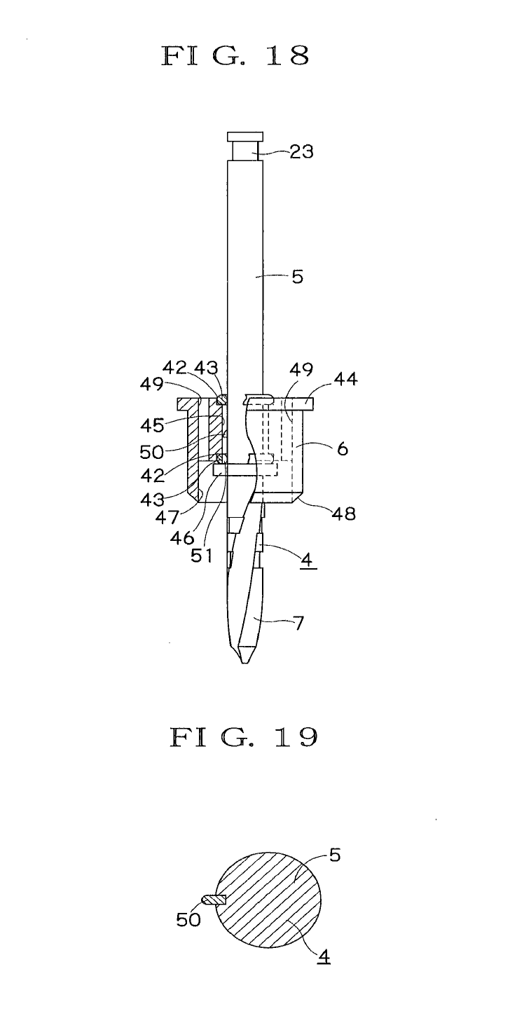

In this embodiment, as shown in FIGS. 17 to 19, the ring guide body 6 is movable in the axial direction and is rotatably attached to the shaft 5 of the drill 4. In this instance, annular grooves 42 are provided at the upper portion and lower portion of the ring guide body 6, and an O-ring 43 made of a frictional resistant material such as nitrile rubber, silicone rubber or urethane rubber is fitted in or stuck to the grooves for fixation. By this O-ring 43, the friction force at the contact surface between the shaft 5 of the drill 4 and the inner face of the ring guide body 6 can be reduced. Instead of the O-ring, a sliding bearing may be provided. Or, without providing such an O-ring 43 or a sliding bearing, the shaft 5 of the drill 4 and the inner face of the ring guide body 6 may be directly engaged in such a manner that these can be movable in the axial direction and rotatable.

The ring guide body 6 is provided with a flange 44, a hole 45 into which the drill shaft 5 is inserted, a hole 47 through which a thick shaft portion 46 of the drill shaft 5 is inserted, a tapered portion 48 and water supply holes 49. In this embodiment, when the ring guide body 6 is attached to the drill shaft 5, a projection 50 formed on the shaft 5 of the drill is inserted through a cutaway 51 formed on the O-ring 43, and the movement of the drill in the axial direction is controlled by positioning the projection 50 within the hole 45 of the ring guide body 6. The projection 50 is installed by inserting a pin into the drill shaft 5 or welding or bonding a projection thereto. This projection 50 may not be provided in some cases. The thick shaft portion 46 of the drill may be integrally formed with the shaft 5 or fixed to the shaft 5 with a screw, etc. so that the installation position of the thick shaft portion can be adjusted.

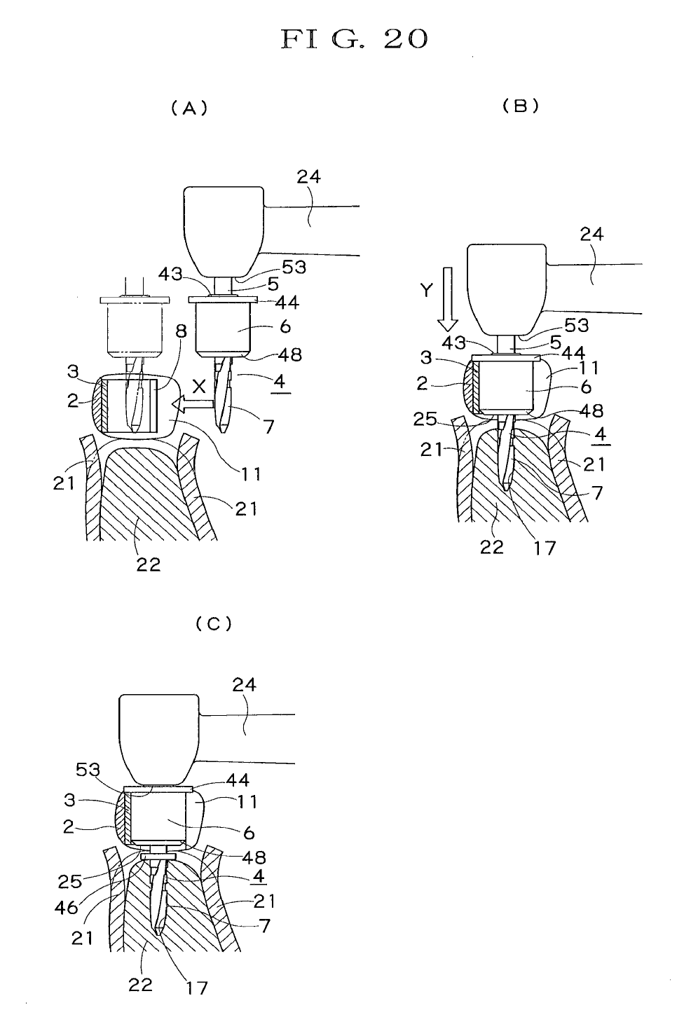

A case where the implant hole 17 is formed by use of the surgical guide 1 having the above guide ring 3 of ellipse configuration and the drill 4 having the ring guide body 6, will be explained with reference to FIG. 20. The parts being same as or equivalent to the members indicated in FIG. 7 are indicated by the same numerals.

At first, as shown in FIG. 20(A), the gum 21 around the implant hole is opened, and the lower jawbone 22 is exposed. The surgical guide 1 is attached in the lower jawbone as shown in FIG. 8. In this instance, the surgical guide 1 is attached to the lower jawbone via adjacent teeth 10, and the lower face of the supporting portion 2 for the implant hole is not in contact with the lower jawbone 22.

In this state, using handpiece (a hole drilling device) 24 in which the upper portion (installation portion 23) of the shaft 5 of the drill 4 is chucked, the drill is moved in the direction of arrow X, and the drill bur 7 is guided into the guide ring 3 through the cutaway 11 of the supporting portion 2 of the surgical guide 1 and the slit 8 of the guide ring 3.

Then, as shown in FIG. 20(B), the drill 4 is moved down in the direction of arrow Y, the ring guide body 6 is guided into the guide ring 3, and the implant hole 17 is formed in the lower jawbone 22 by use of the drill bur 17. When the drill 4 moves down, the ring guide body 6 stops when the flange 44 abuts the guide ring 3, and then the drill 4 subsequently moves downward for drilling.

Subsequently, as shown in FIG. 20(C), the drill 4 is moved down, a lower end face 53 of a handpiece 24 is made in contact with the upper face of the flange 44 of the ring guide body (actually O-ring 43). Since the implant hole 17 is formed in the predetermined depth at this contact position, drilling the hole is stopped at this position. The stopped position can be confirmed through the slit 8 of the guide ring and the cutaway 11 of the surgical guide.

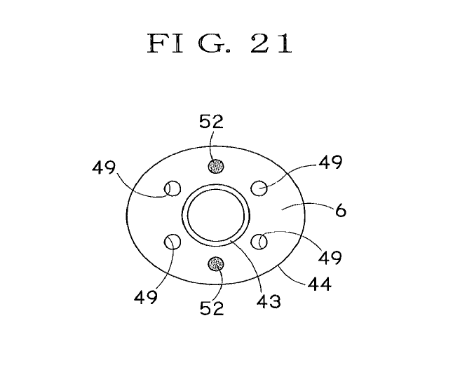

In the ring guide body 6 shown in FIG. 21, a magnet 52 is embedded in the upper portion of the ring guide body 6 as shown in FIG. 16. As the implant hole is sequentially increased in diameter and the drill is replaced, the ring guide body 6 can be temporarily kept at the lower portion of the handpiece by the magnet 52. In this embodiment, two pieces of magnets are provided. However, one magnet or plural magnets other than two may be provided depending on the magnetic force.

### DESCRIPTION OF THE REFERENCE NUMERAL

1 Surgical guide
2 Supporting portion
3 Guide ring
4 Drill
6 Ring guide body
7 Bur
8 Slit
14, 15 Hole
16 Fastener
19 Water supply channel
20, 49 Water supply hole
24 Handpiece
26 Adaptor for implant insertion
27 Implant
43 O-ring
44 Flange
46 Thick shaft portion
52 Magnet

1. A guide system for implant instrument, which comprises a surgical guide having a guide ring, and an implant instrument such as an implant drill or an adaptor for insertion of an implant, wherein a ring guide body is provided on a shaft of the implant instrument and is guided into the guide ring.

2. The guide system for implant instrument according to claim 1, wherein the guide ring is provided with a slit portion on its side face of which the width allows insertion of an implant drill bur or an implant from its lateral side and is smaller than the inner diameter of the guide ring.

3.-8. (canceled)

9. An implant instrument, which comprises an implant instrument such as an implant drill or an adaptor for insertion of an implant, and a ring guide body provided on a shaft of the implant instrument, to be guided into a guide ring of a surgical guide.

10. The implant instrument according to claim 9, wherein a magnet is provided on an upper portion of the ring guide body.

11.-14. (canceled)
